(19) United States
(12) Patent Application Publication
(10) Pub. No.: US 2023/0263467 A1
(43) Pub. Date: Aug. 24, 2023

(54) PROCESSING DEVICE AND METHOD OF HEMODYNAMIC ANALYSIS FOR DETECTING A PARTICULAR SYNDROME

(71) Applicants: Giant Power Technology Biomedical Corp., New Taipei City (TW); National Taipei University of Technology, Taipei City (TW)

(72) Inventors: Po-En Liu, Taipei City (TW); Shu-Hung Chao, New Taipei City (TW); Ming-Kun Huang, New Taipei City (TW); Chien-Jen Wang, New Taipei City (TW); Ing-Lan Liou, New Taipei City (TW); Chun- Young Chang, Taipei City (TW); Chin-Kun Tseng, New Taipei City (TW); Zi-Yi Zhuang, New Taipei City (TW); Ya-Wen Chao, New Taipei City (TW); Hsuan-Yu Liu, New Taipei City (TW); Gu-Neng Wu, New Taipei City (TW); Chun-Ling Lin, New Taipei City (TW); Yuh-Shyan Hwang, Taipei City (TW); San-Fu Wang, New Taipei City (TW); I-Chyn Wey, New Taipei City (TW); Jason King, New Taipei City (TW)

(73) Assignees: Giant Power Technology Biomedical Corp., New Taipei City (TW); National Taipei University of Technology, Taipei City (TW)

(21) Appl. No.: 17/847,373

(22) Filed: Jun. 23, 2022

(30) Foreign Application Priority Data
Feb. 22, 2022 (TW) 111106430

(51) Int. Cl. A61B 5/00; A61B 5/021; A61B 5/024

(52) U.S. Cl. CPC A61B 5/4854; A61B 5/02116; A61B 5/7225; A61B 5/02416; A61B 5/002

(57) ABSTRACT

A method for detecting a particular syndrome based on hemodynamic analysis that includes steps of: obtaining a piece of hemodynamic data representing a hemodynamic waveform; performing moving average (MA) filtering on the hemodynamic waveform to obtain a filtered waveform; determining troughs in order to determine waveform segments of the filtered waveform; determining systolic peaks for determining first and second portions of the waveform segments; determining smoothness of the second portions; and determining a relation between the hemodynamic waveform and a particular syndrome based on the smoothness of the second portions, and generating a detection result.

### CROSS-REFERENCE TO RELATED APPLICATION

This application claims priority of Taiwanese Invention Patent Application No. 111106430, filed on Feb. 22, 2022.

### FIELD

The disclosure relates to hemodynamic analysis, and more particularly to a method and a processing device of hemodynamic analysis for detecting a particular syndrome.

### BACKGROUND

Conventional hemodynamic analysis may be utilized to facilitate detection of certain cardiovascular diseases such as hypertension, atherosclerosis, heart failure, etc.

### SUMMARY

Although hemodynamic analysis is commonly used in modern medicine, the same is not utilized in traditional Chinese medicine. An object of the disclosure is to provide a method and a processing device for hemodynamic analysis that may facilitate detection of syndromes from the perspective of traditional Chinese medicine, such as liver fire blazing and/or heart fire blazing.

Therefore, according to an aspect of the disclosure, a method for detecting a particular syndrome based on hemodynamic analysis is to be performed by a processor. The method includes steps of: obtaining a piece of hemodynamic data that represents a hemodynamic waveform and that is related to a testee; performing first moving average (MA) filtering on the hemodynamic waveform to obtain a first filtered waveform that corresponds to the hemodynamic waveform; using a sliding window algorithm to determine multiple troughs of the first filtered waveform that are each a diastolic nadir of the hemodynamic waveform in order to determine multiple waveform segments of the first filtered waveform that are each between adjacent two of the troughs; for each of the waveform segments, determining a systolic peak within the waveform segment that is a crest nearest a starting point of the waveform segment, so that the waveform segment is composed of a first portion and a second portion, wherein the first portion is from the starting point to the systolic peak of the waveform segment, and the second portion is the rest of the waveform segment; determining smoothness of the second portions of the waveform segments; and determining a relation between the hemodynamic waveform and a particular syndrome based on the smoothness of the second portions of the waveform segments, and generating a detection result indicating a possibility of the testee being afflicted with the particular syndrome based on the relation thus determined.

According to an aspect of the disclosure, a system for detecting a particular syndrome based on hemodynamic analysis includes a hemodynamic sensor and a processing device. The hemodynamic sensor is adapted to be positioned on a testee. The hemodynamic sensor includes a first connection module and a hemodynamic sensing module that is electrically connected to the first connection module. The hemodynamic sensing module is configured to detect a hemodynamic status of the testee in order to generate a piece of hemodynamic data that represents a hemodynamic waveform and that is related to the testee. The processing device is configured to communicate with the hemodynamic sensor. The processing device includes a storage module, a second connection module, a processor and an output module. The storage module stores an application program. The second connection module is configured to communicate with the first connection module. The processor is electrically connected to the storage module and the second connection module. The output module is electrically connected to the processor. The processor is configured to, upon reading and executing the application program stored in the storage module, obtain the piece of hemodynamic data from said hemodynamic sensor through said second connection module. The processor is further configured to perform first moving average (MA) filtering on the hemodynamic waveform to obtain a first filtered waveform that corresponds to the hemodynamic waveform. The processor is further configured to use a sliding window algorithm to determine multiple troughs of the first filtered waveform that are each a diastolic nadir of the hemodynamic waveform, in order to determine multiple waveform segments of the first filtered waveform that are each between adjacent two of the troughs. The processor is further configured to, for each of the waveform segments, determine a systolic peak within the waveform segment that is a crest nearest a starting point of the waveform segment, so that the waveform segment is composed of a first portion and a second portion, wherein the first portion is from the starting point to the systolic peak of the waveform segment, and the second portion is the rest of the waveform segment. The processor is further configured to determine smoothness of the second portions of the waveform segments. The processor is further configured to determine a relation between the hemodynamic waveform and a particular syndrome based on the smoothness of the second portions of the waveform segments. The processor is further configured to generate a detection result indicating a possibility of the testee being afflicted with the particular syndrome based on the relation thus determined. The processor is further configured to control the output module to output the detection result.

### BRIEF DESCRIPTION OF THE DRAWINGS

Other features and advantages of the disclosure will become apparent in the following detailed description of the embodiment (s) with reference to the accompanying drawings, of which:

FIG. 1 is a block diagram that exemplarily illustrates a system according to an embodiment of the disclosure;

FIG. 2 is a flow chart that exemplarily illustrates a method of hemodynamic analysis for detecting a particular syndrome according to an embodiment of the disclosure;

FIG. 3 is a schematic diagram that exemplarily illustrates, according to an embodiment of the disclosure, a waveform segment corresponding to a pulse cycle;

### FIG. 4 is a flow chart that exemplarily illustrates sub-steps of step of the method according to an embodiment of the disclosure;

FIG. 5 is an exemplary schematic diagram of a part of a first filtered waveform that is associated with a healthy person according to an embodiment of the disclosure;

FIG. 6 is an exemplary schematic diagram of a part of the first filtered waveform that is related to liver fire blazing according to an embodiment of the disclosure; and

FIGS. 7-10 are each an exemplary schematic diagram of a part of the first filtered waveform according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

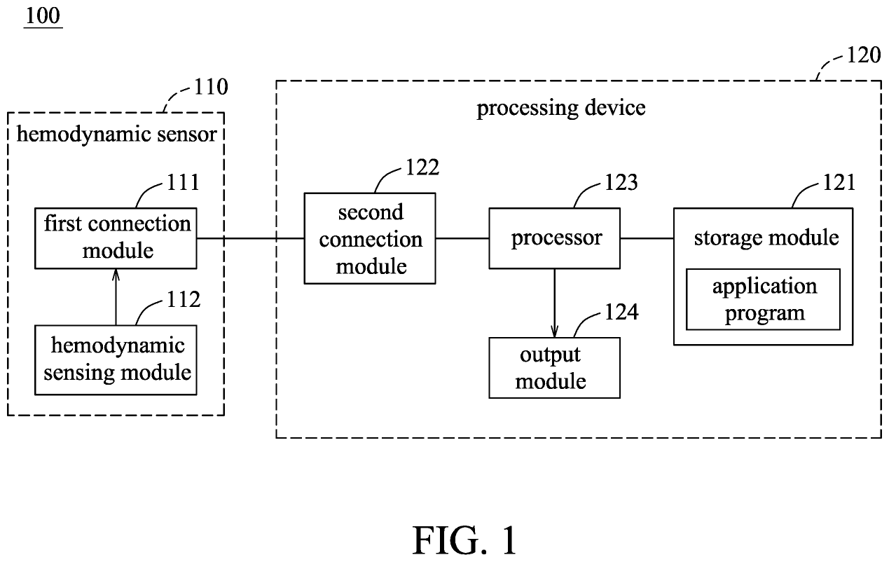

FIG. 1 is a block diagram that exemplarily illustrates, according to an embodiment of the disclosure, a system 100 for detecting a particular syndrome based on hemodynamic analysis. The system 100 includes a hemodynamic sensor 110 and a processing device 120 that are capable of communication with each other. According to some embodiments, the hemodynamic sensor 110 and the processing device 120 may be different devices that are electrically connected to each other, or be integrated into a single device.

The hemodynamic sensor 110 is configured to be wearable by a testee (e.g., on the body of the testee). The hemodynamic sensor 110 includes a first connection module 111, and a hemodynamic sensing module 112 adapted to be positioned on the testee. The first connection module 111 supports at least one communication protocol. The at least one communication protocol may include but is not limited to the Internet Protocol (IP) and/or a short-distance wireless communication protocol such as, for example, a Bluetooth® Protocol or a near-field communication (NFC) protocol. The hemodynamic sensing module 112 is configured to detect a hemodynamic status of the testee in order to generate a piece of hemodynamic data that represents a hemodynamic waveform and that is related to the testee. Specifically, the hemodynamic sensing module 112 is configured to detect the mechanical action of the heart and blood flow of the testee, and to generate hemodynamic data based on the mechanical action thus detected. According to an embodiment of the disclosure, the hemodynamic sensor 110 may be a photoplethysmogram (PPG) sensor, and the hemodynamic data generated by the PPG sensor may be a PPG signal. The hemodynamic sensor 110 is further configured to output the hemodynamic data thus generated to the processing device 120 through the first connection module 111.

The processing device 120 may be a computing system such as a smart phone, a personal computer (PC), a laptop computer, a tablet computer, an ultra-mobile PC (UMPC), a personal digital assistant (PDA), or a cloud server. The processing device 120 includes a storage module 121, a second connection module 122, an output module 124, and a processor 123 that is electrically connected to the storage module 121, the second connection module 122 and the output module 124. The storage module 121 stores an application program. The second connection module 122 also supports the at least one communication protocol (e.g., the Internet Protocol and/or the short-distance wireless communication protocol), and is configured to communicate with the first connection module 111 of the hemodynamic sensor 110 through the at least one communication protocol, so that the processing device 120 may receive the hemodynamic data from the hemodynamic sensor 110 and analyze the hemodynamic data thus received. The processor 123 is configured to implement a method of hemodynamic analysis for detecting a particular syndrome, e.g., liver fire blazing and/or heart fire blazing from the perspective of traditional Chinese medicine, by reading and executing the application program stored in the storage module 121. The output module 124 is controlled by the processor 123 to visually and/or audibly output a detection result generated by the processor 123 implementing the method. In an embodiment where the processing device 120 is a laptop computer, the storage module 121 is the memory, the second connection module 122 is the communication module, the processor 123 is the central processing unit (CPU), and the output module 124 is the screen and the speaker of the laptop computer.

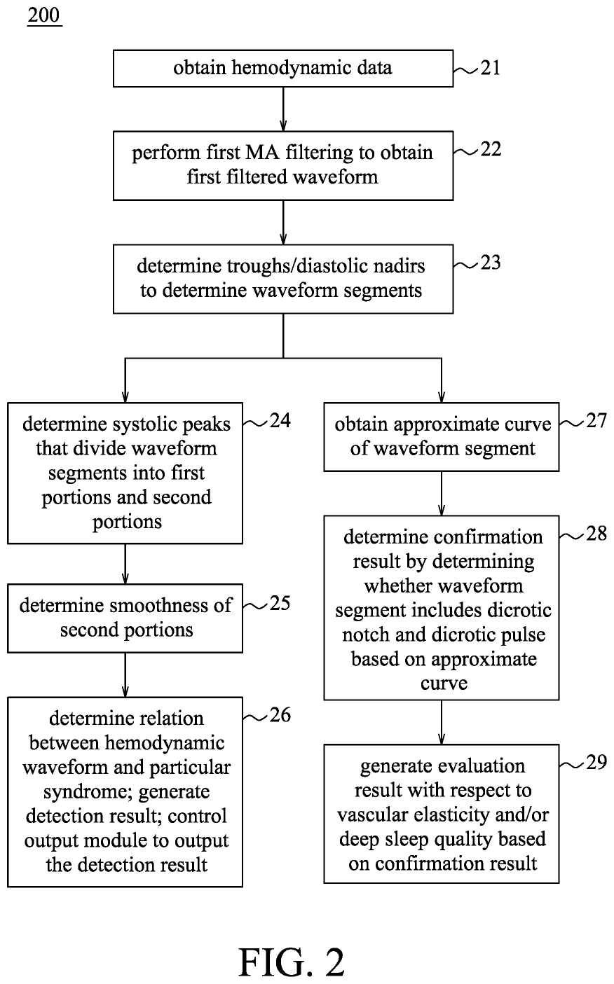

FIG. 2 exemplarily illustrates, according to an embodiment of the disclosure, the method 200 of hemodynamic analysis for detecting the particular syndrome that is to be performed by the processor 123. As shown in FIG. 2, the method 200 includes Steps 21-29.

In Step 21, the processor 123 obtains a piece of hemodynamic data that is generated by the hemodynamic sensor 110 which is worn on a testee. The piece of hemodynamic data represents a hemodynamic waveform and is related to the testee. In an embodiment where the hemodynamic sensor 110 is a PPG sensor, the piece of hemodynamic data obtained by the processor 123 is a PPG signal.

In Step 22, the processor 123 performs first moving average (MA) filtering on the hemodynamic waveform to obtain a first filtered waveform that corresponds to the hemodynamic waveform. In an embodiment, the processor 123 performs the first MA filtering by performing zero-phase digital filtering on the piece of hemodynamic data with an infinite impulse response (IIR) Butterworth bandpass filter, and the first filtered waveform is obtained by using a filtering criterion that is a frequency range from 0.5 Hz to 15 Hz for the Butterworth bandpass filter.

In Step 23, the processor 123 determines multiple troughs of the first filtered waveform that are each a diastolic nadir of the hemodynamic waveform, which is representative of a diastole during a heartbeat interval, in order to determine multiple waveform segments of the first filtered waveform that are each between adjacent two of the troughs and that each correspond to a pulse cycle (also known as cardiac cycle). In particular, in some embodiments, the processor 123 uses a sliding window algorithm to determine the troughs, so as to determine the waveform segments. Specifically, the processor 123 performs the sliding window algorithm by defining a window that has a particular length (e.g., 10 seconds), and applying the window multiple times respectively on multiple portions of the first filtered waveform to find, each time the window is applied on a portion of the first filtered waveform, a lowest one of data points on the first filtered waveform within the window to serve as one of the troughs, wherein the data points each have a first differential value equaling zero. The window is initially applied at the beginning of the first filtered waveform, and then is gradually moved toward the end of the first filtered waveform until the end is reached. It is known that a normal pulse cycle falls within a range between about 0.3 seconds and 1.5 seconds. If the troughs thus determined do not have intervals in said range, the length of the window is adjusted (e.g., reduced by 0.5 seconds) and Step 23 is repeated with the window having the adjusted length.

After Step 23, a first procedure for detecting the particular syndrome and a second procedure for evaluating vascular elasticity and deep sleep quality with respect to last night's sleep are performed. The first procedure includes Steps 24-26, and the second procedure includes Steps 27-29. It is noted that although the first and second procedures are illustrated in FIG. 2 as being performed simultaneously in a multitasking way, the disclosure is not limited thereto. That is, the second procedure may otherwise be performed before or after the first procedure.

In Step 24, the processor 123 determines, for each of the waveform segments, a systolic peak within the waveform segment that is a crest within the waveform segment and nearest a starting point (also referred to as “onset”) of the waveform segment, so that the waveform segment may be divided into a first portion and a second portion based on the systolic peak. Specifically, the first portion is from the onset of the waveform segment to the systolic peak of the waveform segment, and the second portion is the rest of the waveform segment.

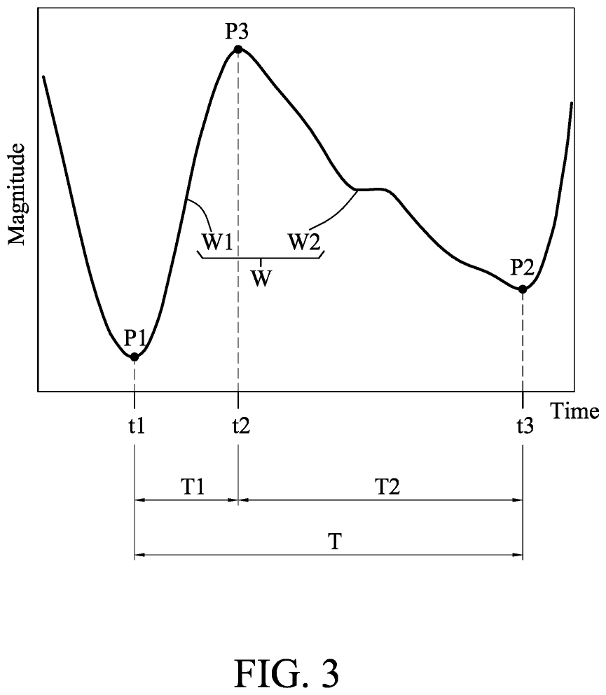

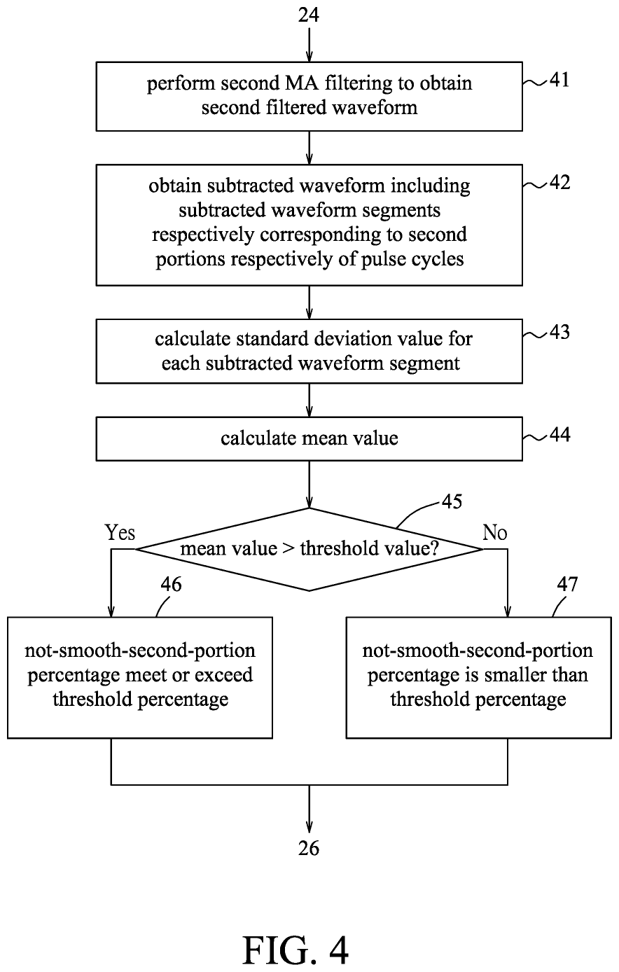

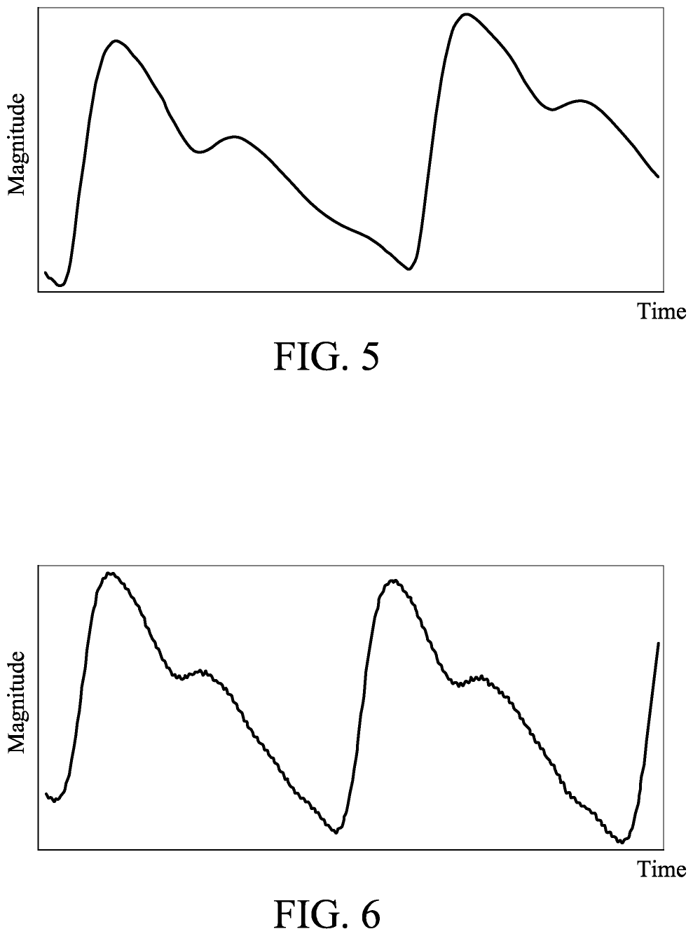

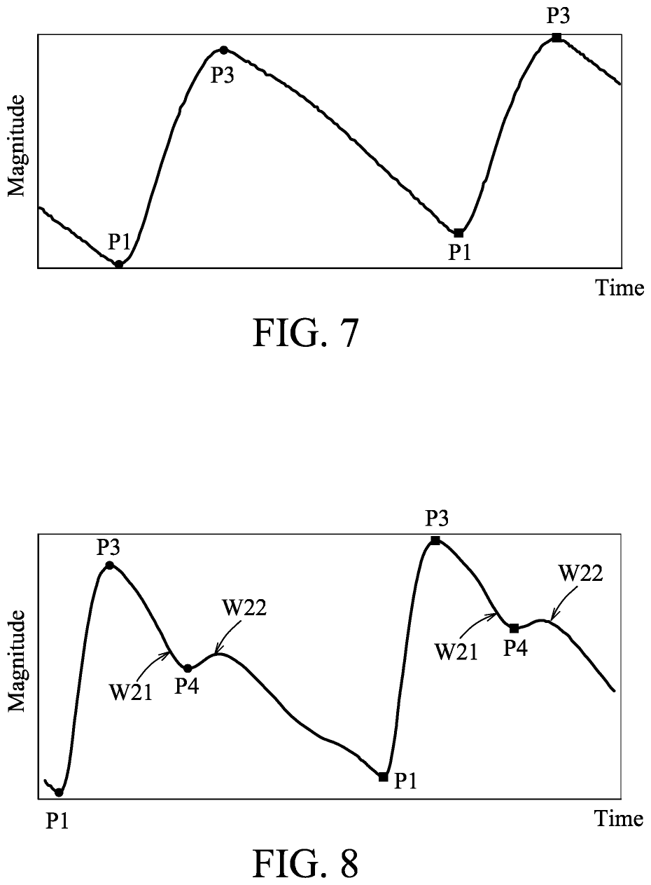

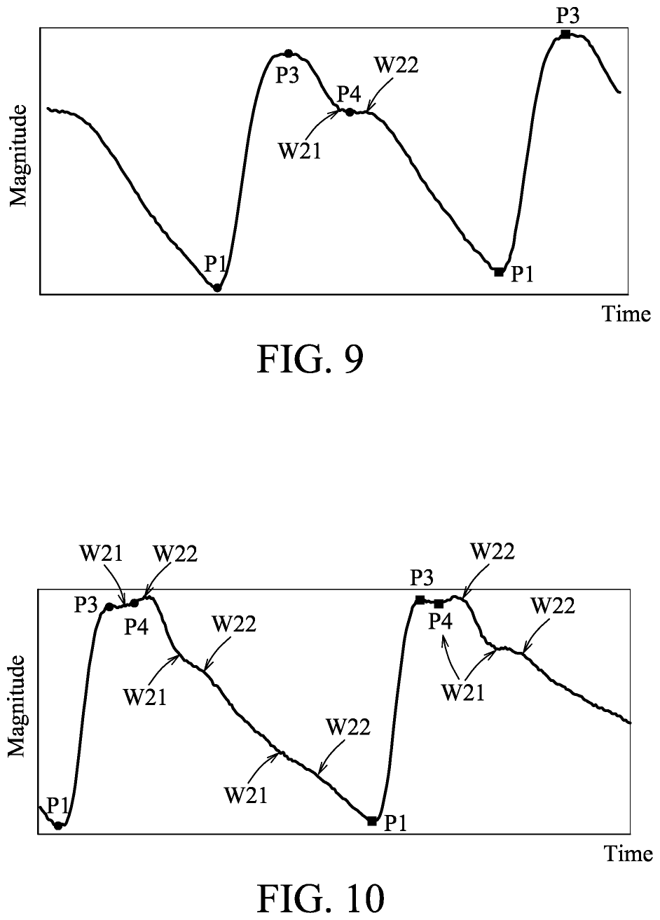

FIG. 3 exemplarily illustrates a waveform segment W (among the waveform segments) that corresponds to a pulse cycle with the onset P1 at time t1, the systolic peak P3 at time t2 and the end P2 at time t3, wherein 5 the onset P1 and the end P2 are adjacent diastolic nadirs, and the systolic peak P3 is the highest data point within the waveform segment W. The waveform segment W is composed of the first portion W1 and the second portion W2. The pulse cycle has a time duration T (from time t1 to time t3) which is composed of a first time duration T1 (from time t1 to time t2) that corresponds to the first portion W1 and a second time duration T2 (from time t2 to time t3) that corresponds to the second portion W2.

In Step 25, the processor 123 determines smoothness of the second portions of the waveform segments. According to some embodiments, Step 25 may include sub-steps 41-47 illustrated in FIG. 4.

Referring to FIG. 4, in Sub-step 41, the processor 123 performs a second MA filtering on the hemodynamic waveform to obtain a second filtered waveform that corresponds to the hemodynamic waveform and that is different from the first filtered waveform obtained in Step 22. The second MA filtering is performed in the same way as the first MA filtering performed in Step 22, but uses a filtering criterion that is different from the filtering criterion used in Step 22. According to some embodiments, when the first filtered waveform is obtained by using a filtering criterion that is a first frequency range, the second filtered waveform may be obtained by using a second frequency range that is wider than the first frequency range. For example, in an embodiment, the first filtered waveform is obtained by using a filtering criterion that is a frequency range from 0.5 Hz to 15 Hz, and the second filtered waveform is obtained by using a frequency range from 0.5 Hz to 100 Hz.

In Sub-step 42, the processor 123 obtains a subtracted waveform by subtracting one of the first filtered waveform and the second filtered waveform from the other of the first filtered waveform and the second filtered waveform. The subtracted waveform includes multiple subtracted waveform segments respectively corresponding to the second portions respectively of the waveform segments of the first filtered waveform.

In Sub-step 43, for each of the subtracted waveform segments of the subtracted waveform, the processor 123 calculates a standard deviation value of magnitudes of data points on the subtracted waveform segment.

In Sub-step 44, the processor 123 calculates a mean value of the standard deviation values calculated for the subtracted waveform segments.

In Sub-step 45, the processor 123 compares the mean value thus calculated with a threshold value, in order to determine whether the mean value exceeds the threshold value.

When it is determined in Sub-step 45 that the mean value exceeds the threshold value, the process goes to Sub-step 46 where the processor 123 determines that a percentage (also referred to as “not-smooth-second-portion percentage”) of the second portions of the waveform segments that are not smooth in the entirety of the second portions of the waveform segments meets or exceeds a threshold percentage (that is, at least the threshold percentage of the second portions of the waveform segments of the first filtered waveform are not smooth); otherwise, the process goes to Sub-step 47 where the processor 123 determines that the not-smooth-second-portion percentage is smaller than the threshold percentage. In an embodiment, the threshold value used in Sub-step 45 is 0.005, and the threshold percentage is 50%, but the disclosure is not limited thereto.

Returning to FIG. 2, after the smoothness of the second portions has been determined in Step 25, in Step 26, the processor 123 determines a relation between the hemodynamic waveform and the particular syndrome based on the smoothness of the second portions of the waveform segments, generates a detection result indicating a possibility of the testee being afflicted with the particular syndrome based on the relation thus determined, and controls the output module 124 to visually and/or audibly output the detection result, thereby facilitating a medical staff in making a diagnostic decision with respect to the testee. Specifically, the processor 123 determines that the hemodynamic waveform is highly related to the particular syndrome when it is determined that the not-smooth-second-portion percentage meets or exceeds the threshold percentage, and subsequently controls the output module 124 to output the detection result that indicates a high possibility of the testee being afflicted with the particular syndrome. On the other hand, the processor 123 determines that the hemodynamic waveform is not highly related to the particular syndrome when it is determined that the not-smooth-second-portion percentage is smaller than the threshold percentage, and subsequently controls the output module 124 to output the detection result that indicates a low possibility of the testee being afflicted with the particular syndrome. According to some embodiments where the processor 123 controls a monitor (not shown in FIG. 1) that is internal or external to the processing device 120, the processor 123 may control the monitor to display a message indicating that the syndrome (of liver fire blazing and/or heart fire blazing) is detected after the processor 123 determines that the hemodynamic waveform is related to the particular syndrome. According to some embodiments where the processor 123 controls a speaker (not shown in FIG. 1) that is internal or external to the processing device 120, the processor 123 may control the speaker to output an audio signal indicating that the syndrome of the particular syndrome is detected after the processor 123 determines that the hemodynamic waveform is related to the particular syndrome. In response to receipt of the message or the audio signal, a Chinese medicine practitioner may be advised to make a diagnosis of liver fire blazing and/or heart fire blazing and decide to use a corresponding treatment or therapy to treat the testee diagnosed with liver fire blazing and/or heart fire blazing.

Part of a first filtered waveform that is derived from a hemodynamic waveform that is related to a healthy person (i.e., someone who does not have liver fire blazing or heart fire blazing) is exemplarily illustrated in FIG. 5. It can be seen that the second portions of the waveform segments of the first filtered waveform shown in FIG. 5 are smooth.

Part of a first filtered waveform that is derived from a hemodynamic waveform that is highly related to liver fire blazing is exemplarily illustrated in FIG. 6. It can be seen that the second portions of the waveform segments of the first filtered waveform shown in FIG. 6 are rugged and not smooth. Incidentally, from the perspective of traditional Chinese medicine, the rugged second portion is related to “taut pulse” and is related to staying up late or lacking good deep sleep for a long time which results in liver fire blazing and/or heart fire blazing.

Reference is now made to the second procedure illustrated in FIG. 2 which includes Steps 27-29 for evaluating vascular elasticity and deep sleep quality with respect to last night's sleep. In Step 27, for each of the waveform segments of the first filtered waveform obtained in Step 22, the processor 123 utilizes the Ramer-Douglas-Peucker algorithm to obtain an approximate curve of the waveform segment. According to some embodiments, the approximate curve may alternatively be obtained by applying the Ramer-Douglas-Peucker algorithm on the second filtered waveform (obtained in Sub-step 41 of Step 25) in place of the first filtered waveform.

In Step 28, the processor 123 determines a confirmation result by, for each of the waveform segments of the first filtered waveform, determining whether the waveform segment includes a dicrotic notch and a dicrotic pulse based on the approximate curve of the waveform segment. According to some embodiments, the dicrotic notch and the dicrotic pulse may also be determined based on an article entitled “A Robust PPG Time Plane Feature Extraction Algorithm for Health Monitoring Application” by Abhishek Chakraborty et al. (e.g., by using an algorithm configured based on the article). The confirmation result may indicate that none of the waveform segments includes the dicrotic notch and the dicrotic pulse, or that at least a portion of the waveform segments includes the dicrotic notch and the dicrotic pulse.

In Step 29, the processor 123 generate an evaluation result with respect to at least one of the vascular elasticity and the deep sleep quality (with respect to last night's sleep) related to the testee based on the confirmation result determined in Step 28, and controls the output module 124 to visually and/or audibly output the evaluation result.

Specifically, when the confirmation result indicates that none of the waveform segments includes the dicrotic notch and the dicrotic pulse, the evaluation result generated by the processor 123 indicates vascular sclerosis (or low vascular elasticity). FIG. 7 exemplarily illustrates a part of a first filtered waveform that is derived from a hemodynamic waveform which is related to vascular sclerosis, with the systolic peaks P3 and the diastolic nadirs P1 being denoted.

On the other hand, when the confirmation result indicates that at least a portion of the waveform segments includes the dicrotic notch and the dicrotic pulse, the processor 123 locates, for each dicrotic notch in the waveform segments, a notch point which is a turning point at the bottom of the dicrotic notch, calculates a mean value of the magnitude(s) of the notch point(s) thus located for the dicrotic notch(es), and evaluates the deep sleep quality based on the mean value thus calculated. In addition, the processor 123 determines, for each dicrotic pulse in the waveform segments, a slope of a leading edge of the dicrotic pulse, and evaluates the vascular elasticity based on the slope(s) thus determined for the dicrotic pulse(s). In some embodiments, the processor 123 compares the mean value of the magnitude(s) of notch point(s) with a predetermined threshold. The processor 123 may determine a poor deep sleep quality when the mean value exceeds the predetermined threshold, and determine a good deep sleep quality otherwise. In some embodiments, the processor 123 determines whether a majority of the slope(s) (e.g., more than 75%) of the leading edge(s) of the dicrotic pulse(s) has a positive value. The processor 123 may determine a good vascular elasticity when a majority of the slope(s) has a positive value (that is, when there are multiple dicrotic pulses, most leading edges thereof are literally rising upward), and determine poor vascular elasticity otherwise.

Each of FIGS. 8 and 9 exemplarily illustrates a part of a first filtered waveform, with the dicrotic notches W21, the dicrotic pulses W22, the diastolic nadirs P1, the systolic peaks P3 and the notch points P4 being denoted. In an embodiment, the processor 123 determines a good deep sleep quality and a good vascular elasticity with respect to the waveform shown in FIG. 8 that has notch points P4 farther from the systolic peaks P3 with respect to magnitude and that has dicrotic pulses W22, each of which has a leading edge that has a positive slope. In an embodiment, the processor 123 determines a poor deep sleep quality and a poor vascular elasticity with respect to the waveform shown in FIG. 9 that has the notch point P4 closer to the systolic peak P3 with respect to magnitude and has the dicrotic pulse W22 which has a leading edge that has a near-zero slope.

According to some embodiments, in Step 29, the processor 123 may further determine, for each of the waveform segments, whether multiple dicrotic notches exist in the waveform segment, and determine a poor deep sleep quality when at least a portion of the waveform segments each include multiple dicrotic notches. FIG. also exemplarily illustrates a part of a first filtered waveform, with the dicrotic notches W21, the dicrotic pulses W22, the diastolic nadirs P1, the systolic peaks P3 and the notch points P4 being denoted.

In an embodiment, the processor 123 determines a poor deep sleep quality and a poor vascular elasticity with respect to the waveform shown in FIG. 10 that has multiple dicrotic notches W21 in a single waveform segment corresponding to a pulse cycle, and that has most of its dicrotic pulses W22 not having a leading edge with a positive slope.

To sum up, by using the method and the system for hemodynamic analysis according to the disclosure, a particular syndrome (e.g., liver/heart fire blazing) from the perspective of traditional Chinese medicine, as well as vascular elasticity and deep sleep quality, can be determined based on a piece of hemodynamic data generated by a hemodynamic sensor, providing a more objective way to make diagnostic decisions. The method and the system can be used not only in medical facilities but also at home, allowing common people to understand his/her health condition, and providing auxiliary information when seeking medical treatment.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for detecting a particular syndrome based on hemodynamic analysis that is to be performed by a processor, the method comprising steps of:
obtaining a piece of hemodynamic data that represents a hemodynamic waveform and that is related to a testee;
performing first moving average (MA) filtering on the hemodynamic waveform to obtain a first filtered waveform that corresponds to the hemodynamic waveform;
using a sliding window algorithm to determine multiple troughs of the first filtered waveform that are each a diastolic nadir of the hemodynamic waveform in order to determine multiple waveform segments of the first filtered waveform that are each between adjacent two of the troughs;
for each of the waveform segments, determining a systolic peak within the waveform segment that is a crest nearest a starting point of the waveform segment, so that the waveform segment is composed of a first portion and a second portion, wherein the first portion is from the starting point to the systolic peak of the waveform segment, and the second portion is the rest of the waveform segment;
determining smoothness of the second portions of the waveform segments; and
determining a relation between the hemodynamic waveform and a particular syndrome based on the smoothness of the second portions of the waveform segments, and generating a detection result indicating a possibility of the testee being afflicted with the particular syndrome based on the relation thus determined.

2. The method of claim 1, wherein the step of determining a relation is to determine a relation between the hemodynamic waveform and one of liver fire blazing and heart fire blazing from the perspective of traditional Chinese medicine.

3. The method of claim 1, wherein the step of determining a relation is to determine that the hemodynamic waveform is highly related to the particular syndrome when it is determined that a percentage of the second portions of the waveform segments that are not smooth in the entirety of the second portions of the waveform segments meets or exceeds a threshold percentage, and to generate, when it is determined that the hemodynamic waveform is highly related to the particular syndrome, the detection result indicating a high possibility of the testee being afflicted with the particular syndrome.

4. The method of claim 3, wherein the step of determining a relation is to determine that the hemodynamic waveform is highly related to the particular syndrome when it is determined that at least fifty percent of the second portions of the waveform segments are not smooth.

5. The method of claim 3, wherein the step of determining smoothness includes sub-steps of:
performing second MA filtering on the hemodynamic waveform to obtain a second filtered waveform that corresponds to the hemodynamic waveform and that is different from the first filtered waveform;
obtaining a subtracted waveform by subtracting one of the first filtered waveform and the second filtered waveform from the other of the first filtered waveform and the second filtered waveform, wherein the subtracted waveform includes multiple subtracted waveform segments respectively corresponding to the second portions respectively of the waveform segments of the first filtered waveform;
for each of the subtracted waveform segments of the subtracted waveform, calculating a standard deviation value;
calculating a mean value of the standard deviation values calculated for the subtracted waveform segments of the subtracted waveform;
comparing the mean value thus calculated with a threshold value;
when the mean value thus calculated exceeds the threshold value, determining that at least the threshold percentage of the second portions of the waveform segments of the first filtered waveform are not smooth.

6. The method of claim 5, wherein the sub-step of comparing the mean value is to compare the mean value with the threshold value of 0.005.

7. The method of claim 5, wherein the sub-step of performing second MA filtering is to perform the second MA filtering by using a filtering criterion that is different from a filtering criterion used in the step of performing first MA filtering.

8. The method of claim 1, wherein the step of obtaining a piece of hemodynamic data is to obtain a photoplethysmogram (PPG) signal.

9. The method of claim 1, further comprising steps of:
for each of the waveform segments of the first filtered waveform, utilizing the Ramer-Douglas-Peucker algorithm to obtain an approximate curve of the waveform segment;
determining a confirmation result by, for each of the waveform segments of the first filtered waveform, determining whether the waveform segment includes a dicrotic notch and a dicrotic pulse based on the approximate curve of the waveform segment; and
generating an evaluation result with respect to at least one of a vascular elasticity and a deep sleep quality related to the testee based on the confirmation result thus determined.

10. The method of claim 1, wherein the step of performing first MA filtering is to perform zero-phase digital filtering on the hemodynamic waveform with a Butterworth bandpass filter.

11. A system for detecting a particular syndrome based on hemodynamic analysis, comprising:
a hemodynamic sensor adapted to be positioned on a testee, said hemodynamic sensor including
a first connection module, and
a hemodynamic sensing module electrically connected to said first connection module, said hemodynamic sensing module being configured to detect a hemodynamic status of the testee in order to generate a piece of hemodynamic data that represents a hemodynamic waveform and that is related to the testee; and
a processing device configured to communicate with said hemodynamic sensor, said processing device including
a storage module storing an application program,
a second connection module configured to communicate with said first connection module,
a processor electrically connected to said storage module and said second connection module , and
an output module electrically connected to said processor;
wherein said processor is configured to, upon reading and executing the application program stored in said storage module,
obtain the piece of hemodynamic data from said hemodynamic sensor through said second connection module,
perform first moving average (MA) filtering on the hemodynamic waveform to obtain a first filtered waveform that corresponds to the hemodynamic waveform;
use a sliding window algorithm to determine multiple troughs of the first filtered waveform that are each a diastolic nadir of the hemodynamic waveform, in order to determine multiple waveform segments of the first filtered waveform that are each between adjacent two of the troughs,
for each of the waveform segments, determine a systolic peak within the waveform segment that is a crest nearest a starting point of the waveform segment, so that the waveform segment is composed of a first portion and a second portion, wherein the first portion is from the starting point to the systolic peak of the waveform segment, and the second portion is the rest of the waveform segment,
determine smoothness of the second portions of the waveform segments,
determine a relation between the hemodynamic waveform and a particular syndrome based on the smoothness of the second portions of the waveform segments,
generate a detection result indicating a possibility of the testee being afflicted with the particular syndrome based on the relation thus determined, and
control said output module to output the detection result.

12. The system of claim 11, wherein said first connection module and said second connection module are configured to communicate with each other via short-range wireless communication.

13. The system of claim 11, wherein said first connection module and said second connection module are configured to communicate with each other via at least one of Bluetooth® or near-field communication (NFC).

14. The system of claim 11, wherein said processor is configured to, upon reading and executing the application program stored in said storage module, determine a relation between the hemodynamic waveform and one of liver fire blazing and heart fire blazing from the perspective of traditional Chinese medicine based on the smoothness of the second portions of the waveform segments.

15. The system of claim 11, wherein said processor is configured to, upon reading and executing the application program stored in said storage module,
determine that the hemodynamic waveform is highly related to the particular syndrome when it is determined that a percentage of the second portions of the waveform segments that are not smooth in the entirety of the second portions of the waveform segments meets or exceeds a threshold percentage; and
generate, when it is determined that the hemodynamic waveform is highly related to the particular syndrome, the detection result indicating a high possibility of the testee being afflicted with the particular syndrome.

16. The system of claim 15, wherein said processor is configured to determine that the hemodynamic waveform is highly related to the particular syndrome when it is determined that at least fifty percent of the second portions of the waveform segments are not smooth.

17. The system of claim 15, wherein said processor is configured to determine the smoothness of the second portions of the waveform segments by performing a smoothness determination procedure including steps of:
performing second MA filtering on the hemodynamic waveform to obtain a second filtered waveform that corresponds to the hemodynamic waveform and that is different from the first filtered waveform;
obtaining a subtracted waveform by subtracting one of the first filtered waveform and the second filtered waveform from the other of the first filtered waveform and the second filtered waveform, wherein the subtracted waveform includes multiple subtracted waveform segments respectively corresponding to the second portions respectively of the waveform segments of the first filtered waveform;
for each of the subtracted waveform segments of the subtracted waveform, calculating a standard deviation value;
calculating a mean value of the standard deviation values calculated for the subtracted waveform segments of the subtracted waveform;
comparing the mean value thus calculated with a threshold value;
when the mean value thus calculated exceeds the threshold value, determining that at least the threshold percentage of the second portions of the waveform segments of the first filtered waveform are not smooth.

18. The system of claim 17, wherein the threshold value is 0.005.

19. The system of claim 17, wherein said processor is configured to perform the second MA filtering by using a filtering criterion that is different from a filtering criterion used for performing the first MA filtering.

20. The system of claim 11, wherein the piece of hemodynamic data is a photoplethysmogram (PPG) signal.

21. The system of claim 11, wherein said processor is further configured to, upon reading and executing the application program stored in said storage module,
for each of the waveform segments of the first filtered waveform, utilize the Ramer-Douglas-Peucker algorithm to obtain an approximate curve of the waveform segment;
determine a confirmation result by, for each of the waveform segments of the first filtered waveform, determining whether the waveform segment includes a dicrotic notch and a dicrotic pulse based on the approximate curve of the waveform segment; and
generate an evaluation result with respect to at least one of a vascular elasticity and a deep sleep quality related to the testee based on the confirmation result thus determined.

22. The system of claim 11, wherein said processor is configured to perform the first MA filtering by performing zero-phase digital filtering on the hemodynamic waveform with a Butterworth bandpass filter.
